(19) United States
(12) Patent Application Publication
(10) Pub. No.: US 2024/0041707 A1
(43) Pub. Date: Feb. 8, 2024

(54) SOLID COMPOSITION COMPRISING A COMBINATION OF PARTICULAR ANIONIC SURFACTANTS AND AT LEAST ONE CATIONIC POLYSACCHARIDE

(71) Applicant: L'OREAL, Paris (FR)

(72) Inventors: Christelle MORVAN, Saint Ouen (FR); Andreia TEIXEIRA, Saint Ouen (FR)

(21) Appl. No.: 18/255,660

(22) PCT Filed: Dec. 3, 2021
(86) PCT No.: PCT/EP2021/084247

§ 371 Date: Jun. 2, 2023

(30) Foreign Application Priority Data
Dec. 3, 2020 (FR) 2012598

(51) Int. Cl. A61K 8/02; A61Q 5/02; A61Q 5/12

(52) U.S. Cl. CPC A61K 8/0216; A61Q 5/02; A61Q 5/12; A61K 2800/87; A61K 2800/596

(57) ABSTRACT

The present invention relates to a solid composition intended in particular for washing and/or conditioning keratin fibres, notably human keratin fibres such as the hair, and which comprises a particular combination of at least two anionic surfactants, of which one is of sulfonate type and the other is of carboxylate type, in the presence of at least one cationic polysaccharide. The invention also relates to a packaging article containing said solid composition, and also to cosmetic processes for treating keratin fibres, in particular human keratin fibres such as the hair, using said solid composition or said packaging article. The invention also relates to the use of said solid composition or of said packaging article for washing and/or conditioning keratin fibres, in particular human keratin fibres such as the hair.

The present invention relates to a solid composition intended in particular for washing and/or conditioning keratin fibres, notably human keratin fibres such as the hair, and which comprises a particular combination of at least two anionic surfactants, of which one is of sulfonate type and the other is of carboxylate type, in the presence of at least one cationic polysaccharide.

The invention also relates to a packaging article containing said solid composition, and also to cosmetic processes for treating keratin fibres, in particular human keratin fibres such as the hair, using said solid composition or said packaging article.

The invention also relates to the use of said solid composition or of said packaging article for washing and/or conditioning keratin fibres, in particular human keratin fibres such as the hair.

In the field of hair hygiene, products for washing keratin fibres are generally intended to cleanse said fibres while at the same time giving them good cosmetic properties. Conventional products, such as shampoos, are usually in more or less thickened liquid form. However, on account of their liquid texture, these products may have various drawbacks, and may notably prove to be difficult to measure out.

The reason for this is that the more liquid they are, the greater their tendency to escape between the fingers, making them difficult to measure out and leading to waste. These products may also escape from their packaging, which is a source of inconvenience to the consumer when these products come into contact with clothing or objects, for example when travelling.

In order to modify the texture of these products, and notably to make it more compact, thickeners are generally used. However, the addition of these compounds usually comes at the expense of the cosmetic effects of the compositions. The use of these thicker compositions moreover necessitates a large amount of rinsing water in order to remove the surplus of product on the fibres. Now, in many countries where access to water is restricted, the rinsing time and consequently the amount of water required to properly rinse off the product are key indicators of the working qualities of a composition.

In order to overcome some of these problems, novel solid cosmetic formulations, notably shampoos in the form of solid granules or powder, have been developed. However, these novel formulations are not always entirely satisfactory. Those which are in loose powder form may, indeed, pose problems of volatility, uptake and/or measuring out, whereas those which are in the form of agglomerates, for instance granules, may have a tendency to disintegrate or break down with difficulty in the presence of water. Thus the latter do not always make it possible to obtain a rapid start of foaming and/or a satisfactory abundance of foam, having a negative impact on their use and their spreading on keratin fibres. They may also be difficult to remove on rinsing and may occasionally even leave residues on the fibres which the consumer finds unpleasant. These formulations may also not be entirely satisfactory in terms of cosmetic performance qualities, notably in terms of suppleness, feel, softness, sheen and disentangling.

Thus, there is a real need to provide a composition in solid form which has an improved environmental profile, i.e. which requires little water throughout its use. The composition must not only be easy to take up, break down easily and have good foaming properties, notably in terms of the start of foaming and the foam abundance and density, but must also rinse out quickly without leaving residues on the keratin fibres.

The composition must also have good detergent power while at the same time affording satisfactory cosmetic properties, notably in terms of suppleness, feel, softness, sheen and disentangling.

It has now been found that a solid composition comprising a particular combination of at least two anionic surfactants, of which one is of sulfonate type and the other is of carboxylate type, in the presence of an amphoteric or zwitterionic surfactant, a filler and a cationic polysaccharide, makes it possible to achieve the objectives presented above, and notably to propose a composition in solid form which combines good detergent power with improved foam properties, without, however, requiring large amounts of water.

One subject of the present invention is a solid composition comprising:
(i) one or more anionic surfactants of sulfonate type,
(ii) one or more anionic surfactants of carboxylate type,
(iii) one or more amphoteric or zwitterionic surfactants,
(iv) one or more fillers,
(v) one or more cationic polysaccharides other than the filler(s) (iv),
the composition comprising a water content of less than 5% by weight, relative to the total weight of the composition.

The particular combination of the compounds of the invention makes it possible to obtain a solid composition that is easy to take up, to handle and to measure out. Specifically, the composition thus obtained has a cohesion or granulation such that the uptake and measuring-out properties are improved. The composition can then be packaged in single-dose form, which is a form that is particularly advantageous, for example, when travelling or performing a sporting activity (lightened bags, limited risks of leakage, reduced waste).

This composition also breaks down rapidly on contact with water and readily and quickly produces a firm, creamy and abundant foam, the quality of which is comparable to that of the foam obtained with a conventional liquid shampoo composition. This foam can then be easily and uniformly distributed on the keratin fibres.

Moreover, the composition of the invention rinses out rapidly without leaving unpleasant residues on the fibres and gives them a natural, clean feel after rinsing. Fibres treated with the composition of the invention also have good cosmetic properties, notably in terms of softness, suppleness and feel. They also have good strand separation and are thus easier to disentangle.

A subject of the present invention is also a cosmetic treatment process, notably for washing and/or conditioning keratin fibres, in particular human keratin fibres such as the hair, comprising the application to said keratin fibres of a solid composition as defined previously, the solid composition being applied directly to said keratin fibres or after having been moistened beforehand with water.

The present invention also relates to the use of a solid composition as defined previously for washing and/or conditioning keratin fibres, in particular human keratin fibres such as the hair.

The present invention also relates to a packaging article comprising:
an envelope defining at least one cavity, the envelope comprising one or more water-soluble and/or liposoluble compounds;
a solid composition as defined above;
it being understood that the solid composition is in one of the cavities defined by the envelope.

This packaging article notably solves the problems of measuring out of the solid composition. It also facilitates its storage and transportation. In particular, the packaging article of the invention affords better protection of the composition against moisture.

The packaging article may also make it possible to obtain a final keratin fibre washing and/or conditioning composition that is more thickened in the hand, which may be in cream form. It may also act as a foam booster. Specifically, the volume of foam obtained after dilution of the packaging article may be greater than the volume of foam obtained after dissolution of the solid composition alone.

The present invention also relates to the use of a packaging article as defined previously for washing and/or conditioning keratin fibres, in particular human keratin fibres such as the hair.

A subject of the present invention is also a cosmetic treatment process, notably for washing and/or conditioning keratin fibres, in particular human keratin fibres such as the hair, comprising a step of using a packaging article as defined above.

Preferably, said cosmetic treatment process comprises the following steps:
i) mixing the packaging article in a composition that is capable of dissolving, totally or partially, the envelope of said packaging article,
ii) applying the composition obtained in step i) to the keratin fibres,
iii) optionally leaving to stand,
iv) rinsing said keratin fibres, and
v) optionally drying said keratin fibres.

Other subjects, characteristics, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

The solid composition according to the present invention comprises a water content of less than 5% by weight, preferably less than 4% by weight, more preferably less than 3% by weight, relative to the total weight of the composition. Even more preferably, the solid composition according to the invention comprises a water content of 0% by weight, relative to the total weight of the composition.

Such a composition will be referred to as an “anhydrous composition” in the following description. In other words, the solid composition according to the present invention corresponds to an anhydrous solid composition in the following description.

In particular, the composition does not comprise any water added during its preparation, the residual water that may be present possibly originating from the starting materials used during the preparation.

The anhydrous solid composition according to the invention may be in powder, paste, particle (for example spherical particles such as small beads or granules), compressed tablet, stick or cake form. Preferably, the composition according to the invention is in the form of a powder or of particles.

The term “powder” means a composition in pulverulent form, which is preferably essentially free of dust (or fine particles). In other words, the particle size distribution of the particles is such that the weight content of particles which have a size of less than or equal to 50 micrometres (content of fines), preferably less than or equal to 45 micrometres (content of fines), is advantageously less than or equal to 5% by weight, preferably less than 3% by weight and more particularly less than 1% by weight, relative to the total weight of the particles (particle size evaluated using a Retsch AS 200 Digit particle size analyser; oscillation height: 1.25 mm/screening time: 5 minutes).

The term “paste” means a composition having a viscosity of greater than 5 poises (0.5 Pa·s), and preferably greater than 10 poises (1 Pa·s), measured at 25° C. and at a shear rate of 1 s⁻¹; this viscosity possibly being determined using a cone-plate rheometer.

The term “particles” means small fractionated objects formed from solid particles that are aggregated together, of variable shapes and sizes. They may be in regular or irregular form. They may in particular be in spherical form (such as granules, granulates or beads) or in square, rectangular or elongated form such as sticks. Spherical particles are most particularly preferred.

Advantageously, the size of the powders or particles is, in its largest dimension, between 45 μm and 5 mm, preferably between 50 μm and 2 mm, more preferentially between 50 μm and 1 mm, and better still between 60 μm and 600 μm.

When the anhydrous solid composition according to the invention is not in powder or particle form, it advantageously has a penetration force at 25° C. and 1 atm of greater than or equal to 200 g, preferably greater than or equal to 300 g, more preferentially greater than or equal to 400 g and better still greater than or equal to 500 g. The penetration force is determined by penetrometry. The texture analysis measurements are performed at 25° C. using a Stable Micro Systems TA.XT Plus texturometer. The penetrometry experiments are performed with a metal rod equipped with a screwed end piece, said end piece being a P/2N needle of 2 mm for the top part, connected to the measuring head. The piston penetrates into the sample at a constant speed of 1 mm/s, to a depth of 5 mm. The force exerted on the piston is recorded and the mean value of the force is calculated.

The anhydrous solid composition according to the invention may be in the form of a compressed anhydrous solid composition, notably compressed using a manual or mechanical press. Preferably, the hardness of the compressed anhydrous solid composition is between 10 and 300 N, more preferentially between 15 and 200 N and better still between 15 and 100 N.

The density of the anhydrous solid composition according to the present invention is preferably between 0.1 and 1, more preferentially between 0.2 and 0.8 and better still between 0.3 and 0.6.

A given amount (mass, m) of powder is placed in a measuring cylinder. The measuring cylinder is then automatically tapped 2500 times. The volume (v) thus obtained is read on the measuring cylinder and the density (d) is then determined according to the formula d=m/v.

The Anionic Surfactants of Sulfonate Type

The anhydrous solid composition according to the present invention comprises one or more anionic surfactants of sulfonate type.

In the sense of the present invention, the term “anionic surfactant of sulfonate type” means an anionic surfactant containing one or more sulfonic or sulfonate functions (—SO₃H or —SO₃), which may optionally contain one or more carboxylic or carboxylate functions (—COOH or —COO⁻), and does not contain any sulfate functions.

Surfactants of this kind may advantageously be chosen from alkylsulfonates, alkylamidesulfonates, alkylarylsulfonates, alpha-olefinsulfonates, paraffinsulfonates, alkylsulfosuccinates, alkylethersulfosuccinates, alkylamidesulfosuccinates, alkylsulfoacetates, sulfolaurates, N-acyltaurates, acylisethionates, and salts thereof and mixtures thereof; the alkyl groups of these compounds contain notably from 8 to 30 carbon atoms, preferably from 8 to 26, and more preferentially from 10 to 22 carbon atoms; the aryl group denotes preferably a phenyl or benzyl group; these compounds may be polyoxyalkylenated, notably polyoxyethylenated and in that case contain preferably from 1 to 50 ethylene oxide units, and more preferentially from 2 to 10 ethylene oxide units.

Preferably, the anionic surfactant(s) of sulfonate type are chosen from N-acyltaurates, and notably N-acyl N-methyltaurates, acylisethionates, and sulfolaurates such as disodium 2-sulfolaurate, and also salts thereof and mixtures thereof.

The anionic surfactant(s) of sulfonate type may more preferentially be chosen advantageously from the compounds of formula (I):

R₁—COX—R₂—SO₃M (I)

formula (I), in which:
R₁ represents a linear or branched, preferably linear, alkyl group comprising from 8 to 30 carbon atoms, preferably from 8 to 26 carbon atoms, and more preferentially from 10 to 22 carbon atoms,
X represents an oxygen atom or a —N(CH₃)— or —NH— group, preferably an oxygen atom,
R₂ represents a linear or branched alkyl group comprising from 1 to 4 carbon atoms, and
M denotes a hydrogen atom, an ammonium ion, an ion obtained from an alkali metal or alkaline-earth metal, or an ion obtained from an organic amine.

The anionic surfactant(s) of sulfonate type, and notably those of formula (I) as defined above, may be used in salified or unsalified form.

Salts which may be used in particular are alkali metal salts, such as the sodium or potassium salts, ammonium salts, amine salts, amino alcohol salts or alkaline-earth metal salts, for example magnesium salts.

Amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferably, the anionic surfactant(s) of sulfonate type are chosen from acylisethionates and mixtures thereof, and more preferentially from acyl(C₈-C₃₀)isethionates and mixtures thereof, which are used in the form of salts, and even better still in the form of alkali metal or alkaline-earth metal salts, and more particularly of sodium or magnesium salts.

Examples of particularly preferred acyl(C₈-C₃₀)isethionate include notably the cocoylisethionates and the lauroyl methyl isethionates, more particularly in the form of sodium salts.

The total content of the anionic surfactant(s) of sulfonate type present in the anhydrous solid composition according to the invention ranges preferably from 1% to 30% by weight, more preferentially from 3% to 25% by weight, better still from 5% to 20% by weight, and even better still from 8% to 16% by weight, relative to the total weight of the composition.

In a preferred variant of the invention, the anionic surfactant(s) of sulfonate type are chosen from acyl(C₈-C₃₀)isethionates and mixtures thereof, and the total content of the acyl(C₈-C₃₀)isethionate (s) present in the anhydrous solid composition according to the invention ranges preferably from 1% to 30% by weight, more preferentially from 3% to 25% by weight, better still from 5% to 20% by weight, and even better still from 8% to 16%, relative to the total weight of the composition.

The Anionic Surfactants of Carboxylate Type

The anhydrous solid composition according to the present invention further comprises one or more anionic surfactants of carboxylate type.

In the sense of the present invention, the term “anionic surfactant of carboxylate type” means an anionic surfactant containing one or more carboxylic or carboxylate functions (—COOH or —COO⁻), which does not contain any sulfonic or sulfonate function (—SO₃H or —SO₃⁻) and does not contain any sulfate function.

Surfactants of these kinds may advantageously be chosen from acyllactates, N-acylglycinates, N-acylsarcosinates and N-acylglutamates, alkyl ether carboxylates, alkyl glucose carboxylates, alkyl glucoside tartrates and alkyl glucoside citrates, where the acyl or alkyl groups contain preferably from 8 to 30 carbon atoms, better still from 10 to 22 carbon atoms; and mixtures thereof; and also the unsalified forms of these compounds.

The anionic surfactant(s) of carboxylate type may preferably be chosen advantageously from the compounds of formula (II):

R—(OCH₂CH₂)ₙW—(CHY₁)ₚ—COOX (II)

formula (II), in which:
Y₁ denotes a hydrogen atom, a group (CH₂)_{q}COOX or a hydroxyl group;
W denotes an oxygen atom, a group (O-Glu-O)ᵣ—(COCH(Y₂)—(C(OH)COOX)ₜ)ₛ or a group CO—NR₃;
Y₂ denotes a hydrogen atom or a hydroxyl group;
R₃ denotes a hydrogen atom or a methyl group;
X denotes a hydrogen atom, an ammonium ion, an ion obtained from an alkali metal or alkaline-earth metal, or an ion obtained from an organic amine;
R denotes a linear or branched, preferably linear, alkyl group comprising from 8 to 30 carbon atoms, preferably from 8 to 26 carbon atoms, and more preferentially from 10 to 22 carbon atoms;
Glu denotes a divalent radical obtained from glucopyranose with removal of 2 hydroxyl groups;
p is equal to 0 or 1;
q denotes an integer ranging from 1 to 10;
n denotes an integer ranging from 0 to 50;
r denotes a number ranging from 1 to 10;
s is equal to 0 or 1; and
t is equal to 0 or 1.

The anionic surfactant(s) of carboxylate type (ii) are preferably chosen from the compounds of formula (II) for which:
Y₁ denotes a hydrogen atom or a group (CH₂)_{q}COOX;
W denotes a group CO—NR₁;
R₃ denotes a hydrogen atom or a methyl group;
X denotes a hydrogen atom, an ammonium ion, an ion obtained from an alkali metal or alkaline-earth metal, or an ion obtained from an organic amine;
R denotes a linear or branched, preferably linear, alkyl group comprising from 8 to 30 carbon atoms, preferably from 8 to 26 carbon atoms, and more preferentially from 10 to 22 carbon atoms;
p is equal to 0 or 1, preferably 0;
q denotes an integer ranging from 1 to 10;
n denotes an integer ranging from 0 to 50.

The anionic surfactant(s) of carboxylate type are more preferentially chosen from the compounds of formula (II) for which:
n=0, p=1, Y₁═H, W═CONH (N-acylglycinates),
n=0, p=1, W═CON(CH₃) and Y₁═H (N-acylsarcosinates), and
n=0, p=1, W═CONH and Y₁═CH₂CH₂COOX (N-acylglutamates).

The anionic surfactant(s) of carboxylate type, and notably those of formula (II) as defined above, may be employed in salified or unsalified form.

As salt it is possible more particularly to use alkali metal salts such as the sodium or potassium salts, ammonium salts, amine salts, amino alcohol salts or alkaline-earth metal salts, for example magnesium salts.

Amino alcohol salts include mono-, di- and triethanolamine salts, mono-, di- or triisopropanolamine salts, 2-amino-2-methyl-1-propanol salts, 2-amino-2-methyl-1,3-propanediol salts and tris(hydroxymethyl)aminomethane salts.

Preference is given to using the alkali metal or alkaline-earth metal salts, and more particularly the sodium or magnesium salts.

The anionic surfactants of carboxylate type are preferentially chosen from N-acyl(C₈-C₃₀) glutamates, and more particularly stearoylglutamates, lauroylglutamates and cocoylglutamates; N-acyl(C₈-C₃₀) sarcosinates, and more particularly palmitoylsarcosinates, stearoylsarcosinates, lauroylsarcosinates and cocoyl-sarcosinates; and mixtures thereof; more particularly in the form of alkali metal or alkaline-earth metal salts, ammonium salts, amine salts or amino alcohol salts.

With particular preference the anionic surfactant(s) of carboxylate type are chosen from N-acyl(C₈-C₃₀) glutamates, more particularly in the form of alkali metal or alkaline-earth metal salts, ammonium salts, amine salts or amino alcohol salts, and mixtures thereof.

The total content of the anionic surfactant(s) of carboxylate type present in the anhydrous solid composition according to the invention ranges preferably from 1% to 40% by weight, more preferentially from 2% to 35% by weight, better still from 5% to 30% by weight, and even better still from 10% to 25% by weight, relative to the total weight of the composition.

In a preferred variant of the invention, the anionic surfactant(s) of carboxylate type are chosen from N-acyl(C₈-C₃₀) glutamates and mixtures thereof, and the total content of the N-acyl(C₈-C₃₀) glutamate (s) present in the anhydrous solid composition according to the invention ranges preferably from 1% to 40% by weight, more preferentially from 2% to 35% by weight, better still from 5% to 30% by weight, and even better still from 10% to 25% by weight, relative to the total weight of the composition.

The weight ratio (R) between the total content of surfactant(s) of carboxylate type (ii) and the total content of surfactant(s) of sulfonate type (i) present in the anhydrous solid composition of the invention is advantageously greater than or equal to 0.6, preferably greater than or equal to 0.7, more preferentially greater than or equal to 0.8, even better still greater than or equal to 1.0, or even strictly greater than 1.0, and better still even greater than or equal to 1.1. Preferably this weight ratio (R) ranges from 0.6 to 5, more preferentially from 0.7 to 4.5, even better still from 0.8 to 4.0, better still even from 1.0 to 3.5, and more preferentially still from 1.1 to 3.0.

In a preferred embodiment, this weight ratio (R) is advantageously greater than or equal to 1, or even strictly greater than 1, this weight ratio (R) ranging preferably from 1 to 5, more preferentially from 1.5 to 3.5, and even better still from 2 to 3.

The anhydrous solid composition according to the invention is preferably free of anionic surfactant of sulfate type.

In the sense of the present invention, the term “anionic surfactant of sulfate type” means surfactants containing at least one group which is anionic or can be ionized to an anionic group, chosen from sulfate functions (—OSO₃H or —OSO₃⁻).

The following anionic surfactants are therefore preferably not present in the anhydrous solid composition according to the invention: alkylsulfate salts, alkylamidosulfate salts, alkylethersulfate salts, alkylamidoethersulfate salts, alkylarylethersulfate salts, and monoglyceride-sulfate salts.

In the sense of the present invention, the term “free of” refers to a composition which does not contain (0%) these anionic surfactants of sulfate type or which contains less than 0.1% by weight of such surfactants, relative to the total weight of the composition.

The total content of the anionic surfactant(s), in other words notably the total content of anionic surfactants of sulfonate type (i) and of anionic surfactants of carboxylate type (ii), present in the anhydrous solid composition according to the invention is advantageously greater than or equal to 15% by weight, this content ranging preferably from 15% to 45% by weight, more preferentially from 20% to 40% by weight, and even better still from 25% to 35% by weight, relative to the total weight of the composition.

The Amphoteric or Zwitterionic Surfactants

The anhydrous solid composition according to the present invention also comprises one or more amphoteric or zwitterionic surfactants.

In particular, the amphoteric or zwitterionic surfactant(s), which are preferably non-silicone, used in the anhydrous solid composition according to the present invention may notably be derivatives of optionally quaternized secondary or tertiary aliphatic amines, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₁-C₆)alkylbetaines and (C₈-C₂₀)alkylamido(C₁-C₆)alkylsulfobetaines, and mixtures thereof.

Among the optionally quaternized derivatives of secondary or tertiary aliphatic amines that may be used, as defined above, mention may also be made of the compounds having the respective structures (III) and (IV) below:

Rₐ—CONHCH₂CH₂—N⁺(R_{b})(R_{c})—CH₂COO⁻,M⁺,X⁻ (III)

in which formula (III):
Rₐ represents a C₁₀ to C₃₀ alkyl or alkenyl group derived from an acid RₐCOOH preferably present in hydrolysed coconut kernel oil; preferably, Rₐ represents a heptyl, nonyl or undecyl group;
R_{b} represents a R-hydroxyethyl group;
Rₑ represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as that chosen from halides, acetates, phosphates, nitrates, (C₁-C₄)alkyl sulfates, (C₁-C₄)alkyl- or (C₁-C₄))alkylarylsulfonates, in particular methyl sulfate and ethyl sulfate; or alternatively M+ and
X⁻ are absent;

Rₐ′—CONHCH₂CH₂—N(B)(B′) (IV)

in which formula (IV):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
Rₐ′ represents a C₁₀ to C₃₀ alkyl or alkenyl group of an acid Rₐ′—COOH which is preferably present in coconut kernel oil or in hydrolysed linseed oil, preferably Rₐ′ an alkyl group, notably a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (V)

R_{a″}—NHCH(Y″)—(CH₂)ₙCONH(CH₂)_{n′}—N(R_{d})(Rₑ) (V)

in which formula (V):
Y″ represents the group —COOH, —COOZ″ or —CH₂—CH(OH)SO₃H or the group CH₂CH(OH)SO₃—Z″;
R_{d} and Rₑ, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀ to C₃₀ alkyl or alkenyl group of an acid R_{a″}—COOH which is preferably present in coconut kernel oil or in hydrolysed linseed oil; and
n and n′ denote, independently of each other, an integer ranging from 1 to 3.

Among the compounds of formula (V), mention may be made of the compound classified in the CTFA dictionary under the name sodium diethylaminopropyl cocoaspartamide and sold by the company Chimex under the name Chimexane HB.

These compounds may be used alone or as mixtures.

Among the amphoteric or zwitterionic surfactants mentioned above, use is advantageously made of (C₈-C₂₀)alkylbetaines, such as cocoyl betaine (C₈-C₂₀)alkylamido(C₃-C₈)alkylbetaines, such as cocamidopropylbetaine, (C₈-C₂₀)alkylamphoacetates, (C₈-C₂₀)alkylamphodiacetates and mixtures thereof; and preferably (C₈-C₂₀)alkylbetaines, (C₈-C₂₀)alkylamido(C₃-C₈)alkylbetaines and mixtures thereof.

Preferentially, the amphoteric or zwitterionic surfactant (s) are chosen from (C₈-C₂₀)alkylbetaines, (C₈-C₂₀)alkylamido(C₃-C₈)alkylbetaines and mixtures thereof, and better still from (C₈-C₂₀)alkylamido(C₃-C₈)alkylbetaines and mixtures thereof.

The total content of the amphoteric or zwitterionic surfactant(s) present in the anhydrous solid composition according to the invention preferably ranges from 1% to 30% by weight, more preferentially from 2% to 25% by weight, and better still from 5% to 20% by weight, relative to the total weight of the composition.

The Fillers

The anhydrous solid composition according to the present invention also comprises one or more fillers.

For the purposes of the present invention, the term “filler” refers to inorganic or organic, polymeric or non-polymeric solid particles.

The fillers according to the invention participate in the dissolution or breakdown of the anhydrous solid composition of the invention, in particular in the presence of water. They may also contribute towards improving the cosmetic performance qualities due to the other compounds present in the composition.

Certain fillers may also have “anticaking” properties.

The inorganic fillers may be chosen from solid salts of alkali metals or alkaline-earth metals, especially sodium or calcium salts, in particular sodium or calcium halides, such as sodium chloride and calcium chloride; or alternatively carbonates, especially sodium or calcium carbonates, for instance calcium carbonate. Mention may also be made of silicates, for instance mica or clays, especially kaolin.

The non-polymeric organic fillers may be chosen from monosaccharides, for instance trehalose, sorbitol and mannitol.

The polymeric organic fillers may be chosen from polysaccharides and mixtures thereof. Mention may be made in particular of cyclodextrins, starches, alginates, gellans, guar gums, celluloses and wood flours. Among the polymeric organic fillers, mention may also be made of crosslinked polyvinylpyrrolidones and polyacrylates (for example Aquakeep).

Advantageously, the filler(s) present in the anhydrous solid composition of the invention are chosen from polymeric organic fillers and mixtures thereof, preferably from cyclodextrins, starches, alginates, gellans, guar gums, celluloses, wood flours, crosslinked polyvinylpyrrolidones, polyacrylates and mixtures thereof, and more preferentially from starches and mixtures thereof.

The total content of the filler(s) present in the anhydrous solid composition of the invention is preferably greater than or equal to 20% by weight, more preferentially greater than or equal to 30% by weight and better still greater than or equal to 35% by weight, relative to the total weight of the composition. Advantageously, the total content of the filler(s) present in the anhydrous solid composition of the invention ranges from 20% to 80% by weight, preferably from 30% to 70% by weight and more preferentially from 35% to 60% by weight, relative to the total weight of the composition.

In a preferred embodiment of the invention, the filler(s) are chosen from starches and mixtures thereof, and the total content of the starch(es) present in the anhydrous solid composition of the invention is preferably greater than or equal to 20% by weight, more preferentially greater than or equal to 30% by weight, better still greater than or equal to 35% by weight, relative to the total weight of the composition.

According to this embodiment, the total content of the starch(es) present in the anhydrous solid composition of the invention advantageously ranges from 20% to 80% by weight, preferably from 30% to 70% by weight and more preferentially from 35% to 60% by weight, relative to the total weight of the composition.

The Cationic Polysaccharides

The anhydrous solid composition according to the present invention also comprises one or more cationic polysaccharides other than the fillers defined above.

For the purposes of the present invention, the term “cationic polysaccharide” means any polysaccharide comprising cationic groups and/or groups that may be ionized into cationic groups. Preferably, the cationic polysaccharides according to the invention do not comprise any anionic groups or any groups that can be ionized into anionic groups.

The cationic polysaccharides may be associative or non-associative.

Preferably, the cationic polysaccharides present in the anhydrous solid composition of the invention are chosen from cationic celluloses, cationic galactomannan gums and mixtures thereof.

Among the cationic polysaccharides which may be used according to the present invention, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polysaccharides sold under the name Ucare Polymer JR (JR 400 LT, JR 125 and JR 30M) or LR (LR 400 and LR 30M) by the company Amerchol. These polysaccharides are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble quaternary ammonium monomer are described notably in patent U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by the company National Starch.

Among the cationic cellulose derivatives, use may also be made of cationic associative celluloses, which may be chosen from quaternized cellulose derivatives, and in particular quaternized celluloses modified with groups including at least one fatty chain, such as linear or branched alkyl groups, linear or branched arylalkyl groups, and linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, and linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (VI):

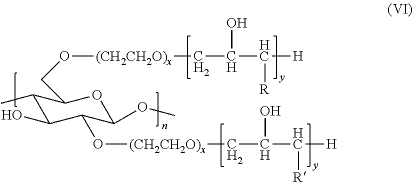

in which:
R represents an ammonium group RaRbRcN⁺—, Q⁻ in which Ra, Rb and Rc, which may be identical or different, represent a hydrogen atom or a linear or branched C₁ to C₃₀ alkyl group, preferably an alkyl group, and Q⁻ represents an anionic counterion such as a halide, for instance a chloride or bromide;
R′ represents an ammonium group R′aR′bR′cN⁺—, Q′⁻ in which R′a, R′b and R′c, which may be identical or different, represent a hydrogen atom or a linear or branched C₁ to C₃₀ alkyl group, preferably an alkyl group, and Q′⁻ represents an anionic counterion such as a halide, for instance a chloride or bromide;
it being understood that at least one of the radicals Ra, Rb, Rc, R′a, R′b and R′c represents a linear or branched C₈ to C₃₀ alkyl group;
n, x and y, which may be identical or different, represent an integer between 1 and 10 000.

Preferably, in formula (VI), at least one of the radicals Ra, Rb, Rc, R′a, R′b or R′c represents a linear or branched C₈ to C₃₀, better still C₁₀ to C₂₄ or even C₁₀ to C₁₄, alkyl group; mention may be made in particular of the dodecyl radical (C₁₂). Preferably, the other radical(s) represent a linear or branched C₁-C₄ alkyl group, notably a methyl.

Preferably, in formula (VI), only one of the radicals Ra, Rb, Rc, R′a, R′b or R′c represents a linear or branched C₈ to C₃₀, better still C₁₀ to C₂₄ or even C₁₀ to C₁₄, alkyl group; mention may be made in particular of the dodecyl radical (C₁₂). Preferably, the other radicals represent a linear or branched C₁ to C₄ alkyl group, notably a methyl.

Advantageously, R may be a group chosen from —N⁺(CH₃)₃, Q′⁻ and —N⁺(C₁₂H₂₅) (CH₃)₂, Q′⁻, preferably a group —N⁺(CH₃)₃, Q′⁻.

Advantageously, R′ may be a group —N⁺(C₁₂H₂₅) (CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200©, sold by the company Amerchol/Dow Chemical;
PG-Hydroxyethylcellulose Cocodimonium Chloride, such as the product Crodacel QM®, sold by the company Croda;
PG-Hydroxyethylcellulose Lauryldimonium Chloride (C₁₂ alkyl), such as the product Crodacel QL®, sold by the company Croda; and
PG-Hydroxyethylcellulose Stearyldimonium Chloride (Cis alkyl), such as the product Crodacel QS®, sold by the company Croda.

Mention may also be made of the hydroxyethylcelluloses of formula (VI) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₁₂H₂₅)N⁺—, Cl⁻. This type of polymer is known under the INCI name Polyquaternium-67. As commercial products, mention may be made of the Softcat Polymer SL® polymers, such as SL-100, SL-60, SL-30 and SL-5, from the company Amerchol/Dow Chemical.

More particularly, the polymers of formula (VI) are, for example, those whose viscosity is between 2000 and 3000 cPs inclusive (between 2 and 3 Pa·s), preferentially between 2700 and 2800 cPs (between 2.7 and 2.8 Pa·s). Typically, Softcat Polymer SL-5 has a viscosity of 2500 cPs (2.5 Pa·s), Softcat Polymer SL-30 has a viscosity of 2700 cPs, Softcat Polymer SL-60 has a viscosity of 2700 cPs (2.7 Pa·s) and Softcat Polymer SL-100 has a viscosity of 2800 cPs (2.8 Pa·s). Use may also be made of Softcat Polymer SX-1300X with a viscosity of between 1000 and 2000 cPs (between 1 and 2 Pa·s).

The cationic galactomannan gums which may be used are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Such products are notably sold under the names Jaguar C13 S, Jaguar C 15, Jaguar C 17 and Jaguar C162 by the company Rhodia.

Preferably, the cationic polysaccharide(s) are chosen from cationic galactomannan gums and mixtures thereof, and more preferentially from cationic guar gums and mixtures thereof.

The total content of the cationic polysaccharide(s) present in the anhydrous solid composition according to the invention is preferably greater than or equal to 0.05% by weight, more preferentially ranges from 0.05% to 5% by weight, better still from 0.1% to 2% by weight, and even more preferentially from 0.2% to 1.5% by weight, relative to the total weight of the composition.

The anhydrous solid composition according to the present invention may optionally also comprise one or more cationic polymers other than the cationic polysaccharides defined above.

The Nonionic Surfactants

The anhydrous solid composition according to the present invention may optionally further comprise one or more nonionic surfactants.

Examples of nonionic surfactants which can be used in the compositions of the present invention are described for example in “Handbook of Surfactants” by M. R. Porter, published by Blackie & Son (Glasgow and London), 1991, pp. 116-178. They are chosen notably from alcohols, alpha-diols, alkyl(C₁-C₂₀)phenols or fatty acids, these compounds being polyethoxylated, polypropoxylated or polyglycerolated, and having at least one fatty chain containing, for example, from 8 to 18 carbon atoms, it being possible for the number of ethylene oxide or propylene oxide groups to range notably from 1 to 100 and for the number of glycerol groups to range notably from 1 to 30.

Mention may also be made of condensates of ethylene oxide and propylene oxide with fatty alcohols; polyethoxylated fatty amides having preferably from 1 to 30 ethylene oxide units, polyglycerolated fatty amides containing on average from 1 to 5 glycerol groups and more particularly from 1.5 to 4, ethoxylated sorbitan fatty acid esters having from 2 to 30 ethylene oxide units, sucrose fatty acid esters, polyethylene glycol fatty acid esters, (C₆ to C₂₄ alkyl)polyglycosides, N—(C₆ to C₂₄ alkyl)glucamine derivatives, and amine oxides such as (C₁₀ to C₁₄ alkyl)amine oxides or N—(C₁₀ to C₁₄ acyl)aminopropylmorpholine oxides.

Among the nonionic surfactants, preference is given more particularly to using alkyl(poly)glycoside nonionic surfactants.

The term “alkyl(poly)glycoside” denotes an alkylpolyglycoside or an alkylmonoglycoside, also called alkylglycoside in the present patent application, which may be alkoxylated by one or more preferably C₂ to C₄ alkylene oxide groups.

The alkyl(poly)glycoside nonionic surfactant(s), used alone or in (a) mixture(s), in accordance with the present invention may be represented by the formula (VII) below:

R₁O—(R₂O)ₜ(G)ᵥ (VII)

formula (VII), in which:
R₁ represents a saturated or unsaturated, linear or branched alkyl group containing from 8 to 24 carbon atoms or an alkylphenyl group in which the linear or branched alkyl group contains from 8 to 24 carbon atoms,
R₂ represents an alkylene group containing approximately from 2 to 4 carbon atoms,
G represents a saccharide unit containing from 5 to 6 carbon atoms,
t denotes a value ranging from 0 to 10, preferably 0 to 4, and
v denotes a value ranging from 1 to 15.

The alkyl(poly)glycoside nonionic surfactant(s) preferably conform to the formula (VII) in which:
R₁ denotes a saturated or unsaturated, linear or branched alkyl group containing from 8 to 18 carbon atoms,
G denotes glucose, fructose or galactose, and preferably glucose,
t denotes a value ranging from 0 to 3, and is preferably equal to 0, and
R₂ and v are as defined above.

The degree of polymerization of the alkyl(poly)glycoside nonionic surfactant(s), as represented for example by the index v in the formula (VII) above, varies on average from 1 to 15, and preferably from 1 to 4. This degree of polymerization varies more particularly from 1 to 2, and even better still from 1.1 to 1.5, on average.

The glycoside bonds between the saccharide units are 1,6 or 1,4, and preferably 1,4.

The alkyl(poly)glycoside nonionic surfactants which may be used in the present invention are preferably alkyl(poly)glucosides notably represented by the products sold by Cognis under the names Plantaren® (600 CS/U, 1200 and 2000) or Plantacare® (818, 1200 and 2000). Use may also be made of the products sold by Seppic under the names Triton CG 110 (or Oramix CG 110) and Triton CG 312 (or Oramix® NS 10), the products sold by BASF under the name Lutensol GD 70 or else those sold by Chem Y under the name AG10 LK, or the products sold by Evonik Goldschmidt under the trade names Tego Care CG 90 or Tego Care GC 90 MB.

As nonionic surfactant it is possible with preference to use the compounds with INCI name caprylyl/capryl glucoside, decyl glucoside, coco glucoside, lauryl glucoside, myristyl glucoside, cetearyl glucoside and/or arachidyl glucoside. The compound with INCI name cetearyl glucoside is particularly preferred.

The total content of the nonionic surfactant(s), when they are present in the anhydrous solid composition according to the invention, ranges preferably from 0.1% to 10% by weight, and more preferentially from 0.2% to 5% by weight, relative to the total weight of the composition.

The anhydrous solid composition according to the invention may preferably comprise one or more nonionic surfactants chosen from alkyl(poly)glycosides and mixtures thereof, and more preferentially one or more nonionic surfactants chosen from alkyl(poly)glucosides and mixtures thereof. According to this embodiment, the total content of alkyl(poly)glycoside(s), preferably of alkyl(poly)glucoside(s), preferably ranges from 0.1% to 10% by weight, and more preferentially from 0.2% to 5% by weight, relative to the total weight of the composition.

The total content of surfactants present in the anhydrous solid composition according to the invention is preferably less than or equal to 60% by weight; more preferentially this total content ranges from 20% to 55% by weight, better still from 30% to 50%, and even better still from 35% to 45% by weight, relative to the total weight of the composition.

The Organic Solvents

The anhydrous solid composition according to the present invention may optionally further comprise one or more organic solvents.

Preferably, the organic solvent(s) are chosen from linear or branched monoalcohols containing from 1 to 8 carbon atoms, and more preferentially from 1 to 4 carbon atoms, polyols, containing notably from 2 to 8 carbon atoms, polyethylene glycols, aromatic alcohols, and mixtures thereof.

As examples of organic solvents that may be used according to the invention, mention may notably be made of ethanol, propanol, butanol, isopropanol, isobutanol, propylene glycol, dipropylene glycol, isoprene glycol, butylene glycol, glycerol, benzyl alcohol and phenoxyethanol, and mixtures thereof.

The organic solvent (s) that may be used according to the invention may be chosen from linear or branched monoalcohols containing from 1 to 4 carbon atoms, and mixtures thereof, preferably from ethanol, propanol, butanol, isopropanol, isobutanol, and mixtures thereof.

Preferably, the organic solvent(s) are chosen from polyols, containing notably from 2 to 8 carbon atoms, and mixtures thereof, and more preferentially from glycerol, propylene glycol, and mixtures thereof.

The total content of the organic solvent(s), when they are present in the anhydrous solid composition according to the invention, is preferably less than or equal to 20% by weight, more preferentially less than or equal to 15% by weight, and better still ranges from 0.5% to 12% by weight, relative to the total weight of the composition.

The anhydrous solid composition according to the present invention may optionally further comprise one or more additional compounds, different from the compounds defined above, and preferably chosen from cationic surfactants, anionic, nonionic, amphoteric and zwitterionic polymers and mixtures thereof, antioxidants, penetrants, sequestrants, fragrances, buffers, dispersants, conditioning agents such as, for example, volatile or non-volatile, modified or non-modified silicones, film formers, ceramides, preservatives, opacifiers, lubricants (or anti-caking agents) and mixtures thereof.

According to a particular embodiment, the anhydrous solid composition of the invention may optionally comprise one or more lubricants. Such a lubricant may notably act as an anti-caking agent.

The lubricant(s) which may be used are different from the fillers and the cationic polysaccharides defined above.

The lubricants which may be used in the anhydrous solid composition of the invention notably include silica, more particular anhydrous colloidal silica, sericite, polyamide (Nylon®) powders, poly-p-alanine and polyethylene powders, powders of tetrafluoroethylene polymers (Teflon®), acrylate-dimethicone copolymers, stearic acid, metal soaps derived from organic carboxylic acids having from 8 to 22 carbon atoms, preferably from 12 to 18 carbon atoms, such as, for example, zinc, magnesium or lithium stearates, zinc laurate and magnesium myristate, alkali metal or alkaline-earth metal carbonates, such as, for example, magnesium, sodium and calcium carbonates, fatty acids such as stearic acid, celluloses, notably crystalline celluloses, and mixtures thereof.

According to one embodiment, the lubricant or lubricants are advantageously chosen from metal soaps derived from organic carboxylic acids having from 8 to 22 carbon atoms, preferably from 12 to 18 carbon atoms, and mixtures thereof, even better still from zinc stearate, magnesium stearate, lithium stearate, zinc laurate, magnesium myristate and mixtures thereof. The lubricant more preferentially is magnesium stearate.

According to another embodiment, the lubricant or lubricants are advantageously chosen from alkali metal or alkaline-earth metal carbonates and mixtures thereof, preferably from magnesium carbonate, sodium carbonate, calcium carbonate and mixtures thereof; and more preferentially magnesium carbonate.

Preferably, when the additional compound or compounds above are present in the anhydrous solid composition according to the invention, the additional compound or compounds are present in general in an amount each of between 0.01% and 20% by weight, relative to the weight of the composition.

Needless to say, a person skilled in the art will take care to select this or these optional additional compounds such that the advantageous properties intrinsically associated with the anhydrous solid composition of the invention are not, or are not substantially, adversely affected by the envisaged addition(s).

A subject of the present invention is also a cosmetic treatment process, and notably a process for washing and/or conditioning keratin fibres, in particular human keratin fibres such as the hair, comprising the application to said keratin fibres of a solid composition as defined previously, the solid composition being applied directly to said keratin fibres or after having been moistened beforehand with water.

The anhydrous solid composition according to the invention may be applied to dry or wet keratin fibres, preferably to wet keratin fibres.

The anhydrous solid composition thus applied may optionally be rinsed off or left on, after an optional leave-on time that may range from 1 to 15 minutes, preferably from 2 to 10 minutes.

Preferably, the anhydrous solid composition is rinsed off after application.

According to a first embodiment of the invention, the anhydrous solid composition is applied directly to the keratin fibres, i.e. without being moistened and/or broken down in water beforehand.

When, according to this first embodiment, the anhydrous solid composition of the invention is applied directly (i.e. without being moistened or broken down beforehand) to the dry keratin fibres, water may optionally be added to said fibres in order subsequently to rub/massage so as to dissolve/pre-emulsify said composition and to form an immediate abundant foam. The foam thus obtained can subsequently be rinsed out after an optional leave-on time.

Conversely, the anhydrous solid composition of the invention may also be applied directly (i.e. without moistening or breaking down beforehand) to the wet keratin fibres, followed by massaging/rubbing to break down the particles and to obtain an immediate abundant foam. The foam thus obtained can subsequently be rinsed out after an optional leave-on time.

According to another embodiment of the invention, the anhydrous solid composition is moistened and/or broken down beforehand in water before being applied to the keratin fibres. According to this embodiment, a small amount (preferably ranging from 1 to 3 g) of anhydrous solid composition is advantageously taken up and dissolved with water, for example in the hand, so as to form an immediate abundant foam. The foam thus obtained may then be applied to the wet or dry keratin fibres, before being optionally rinsed out with water after an optional leave-on time.

A subject of the present invention is also the use of a solid composition as defined previously for washing and/or conditioning keratin fibres, in particular human keratin fibres such as the hair.

The present invention also relates to a packaging article, preferably a cosmetic packaging article, comprising:
an envelope defining at least one cavity, the envelope comprising one or more water-soluble and/or liposoluble compounds,
a solid composition as defined above;
it being understood that the solid composition is in one of the cavities defined by the envelope.

The term “cosmetic packaging article” means an article that is suitable for cosmetic use; in particular for use of the packaging article on keratin fibres, notably the hair, and/or on the scalp. In particular, the packaging article makes it possible to wash and/or condition the keratin fibres, in particular human keratin fibres such as the hair.

Preferably, the packaging article according to the invention is water-soluble or liposoluble at a temperature of less than or equal to 35° C.

Preferably, the envelope of the packaging article according to the invention is water-soluble at a temperature of less than or equal to 35° C.

The term “water-soluble” means soluble in water, in particular in a proportion of at least 10 grams per litre of water, preferably at least 20 g/l, better still at least 50 g/l, at a temperature of less than or equal to 35° C. Thus, when water preferably having a temperature of less than 35° C. is added to the packaging article, the envelope dissolves and releases the anhydrous solid composition present in one of the cavities of the envelope.

The term “liposoluble” means soluble in a liquid fatty substance as defined below, in particular in a proportion of at least 10 grams per litre of liquid fatty substance, in particular in a plant or mineral oil such as liquid petroleum jelly, preferably at least 20 g/l in a liquid fatty substance, better still at least 50 g/l in a fatty substance, at a temperature of less than or equal to 35° C.

The term “temperature of less than or equal to 35° C.” means a temperature not exceeding 35° C. but greater than or equal to 0° C., for example ranging from more than 1 to 35° C., preferably from 5 to 30° C., more preferentially from 10 to 30° C. and better still from 15 to 25° C. It is understood that all the temperatures are given at atmospheric pressure (1 atm).

The packaging article may comprise one or more cavities, at least one of which contains the anhydrous solid composition as defined previously. Preferably, the packaging article comprises only one cavity in which the anhydrous solid composition is contained.

Advantageously, the envelope represents from 0.5% to 20% by weight, preferably from 1% to 15% by weight, more preferentially from 2% to 10% by weight and better still from 4% to 8% by weight relative to the total weight of the packaging article.

Advantageously, the anhydrous solid composition as defined previously represents from 80% to 99.5% by weight, preferably from 85% to 99% by weight, more preferentially from 90% to 98% by weight and better still from 92% to 96% by weight relative to the total weight of the packaging article.

The weight ratio between the total weight of the anhydrous solid composition of the invention and the total weight of the envelope advantageously ranges from 80/20 to 99/1, preferably from 85/15 to 98/2 and more preferentially from 90/10 to 97/3.

The envelope of the packaging article comprises one or more water-soluble and/or liposoluble compounds, preferably one or more water-soluble compounds advantageously chosen from water-soluble polymers and mixtures thereof.

The water-soluble polymer(s) that may be used according to the present invention contain water-soluble units in their backbones. The water-soluble units are obtained from one or more water-soluble monomers.

The term “water-soluble monomer” means a monomer whose solubility in water is greater than or equal to 1%, preferably greater than or equal to 5%, at 25° C. and at atmospheric pressure (760 mmHg).

Said water-soluble polymer(s) that are capable of forming the envelope are advantageously obtained from water-soluble monomers including at least one double bond. These monomers may be chosen from cationic, anionic and nonionic monomers, and mixtures thereof.

As water-soluble monomers that may be used as precursors for the water-soluble units, alone or as a mixture, examples that may be mentioned include the following monomers, which may be in free or salified form:
(meth)acrylic acid,
styrenesulfonic acid,
vinylsulfonic acid and (meth)allylsulfonic acid,
vinylphosphonic acid,
N-vinylacetamide and N-methyl-N-vinylacetamide,
N-vinylformamide and N-methyl-N-vinylformamide,
N-vinyllactams including a cyclic alkyl group containing from 4 to 9 carbon atoms, such as N-vinylpyrrolidone, N-butyrolactam and N-vinylcaprolactam,
maleic anhydride,
itaconic acid,
vinyl alcohol of formula CH₂═CHOH,
vinyl acetate of formula CH₂═CHOC(O)CH₃,
vinyl ethers of formula CH₂═CHOR in which R is a linear or branched, saturated or unsaturated hydrocarbon-based radical containing from 1 to 6 carbon atoms;
dimethyldiallylammonium halides (chloride),
quaternized dimethylaminomethyl methacrylate (DMAEMA),
(meth)acrylamidopropyltrimethylammonium halides (chloride) (APTAC and MAPTAC),
methylvinylimidazolium halides (chloride),
2-vinylpyridine and 4-vinylpyridine,
acrylonitrile,
glycidyl (meth)acrylate,
vinyl halides (chloride) and vinylidene chloride,
the vinyl monomers having the following formula: H₂C═C(R)—C(O)—X, in which:
   R is chosen from H, (C₁-C₆)alkyl such as methyl, ethyl and propyl, and
   X is chosen from:
      alkoxy groups of the type —OR′ in which R′ is a linear or branched, saturated or unsaturated hydrocarbon-based radical containing from 1 to 6 carbons, optionally substituted with at least one halogen (iodine, bromine, chlorine or fluorine); a group from among sulfonic (—SO₃⁻), sulfate (SO₄⁻), phosphate (—PO₄H₂); hydroxyl (—OH); primary amine (—NH₂); secondary amine (NHR₆), tertiary amine (—NR₆R₇) or quaternary amine (—N+R₆R₇R₈) with R₆, R₇ and R₈ being, independently of each other, a linear or branched, saturated or unsaturated hydrocarbon-based radical containing 1 to 6 carbon atoms, with the proviso that the sum of the carbon atoms of R′+R₆+R₇+R₈ does not exceed 6;
      groups —NH₂, —NHR′ and —NR′R″ in which R′ and R″ are, independently of each other, linear or branched, saturated or unsaturated hydrocarbon-based radicals containing from 1 to 6 carbons, with the proviso that the total number of carbon atoms of R′+R″ does not exceed 6, said radicals R′ and R″ being optionally substituted with a halogen (iodine, bromine, chlorine or fluorine); a group from among hydroxyl (—OH); sulfonic (—SO₃—), sulfate (SO₄⁻), phosphate (—PO₄H₂); primary amine (—NH₂); secondary amine (NHR₆), tertiary amine (—NR₆R₇) and/or quaternary amine (—N⁺R₆R₇R₈) with R₆, R₇ and R₈ being, independently of each other, a linear or branched, saturated or unsaturated hydrocarbon-based radical containing 1 to 6 carbon atoms, with the proviso that the sum of the carbon atoms of R′+R″+R₆+R₇+R₈ does not exceed 6. As compounds corresponding to this formula, examples that may be mentioned include N,N-dimethylacrylamide and N,N-diethylacrylamide;
and mixtures thereof.

Anionic monomers that may notably be mentioned include (meth)acrylic acid, acrylamido-2-methylpropanesulfonic acid, itaconic acid and the salts thereof with an alkali metal, an alkaline-earth metal or ammonium or those derived from an organic amine such as an alkanolamine.

Nonionic monomers that may notably be mentioned include (meth)acrylamide, N-vinylformamide, N-vinylacetamide and hydroxypropyl (meth)acrylate, vinyl alcohol of formula CH₂═CHOH, and vinyl acetate of formula CH₂═CHOC(O)CH₃.

The cationic monomers are preferably chosen from quaternary ammonium salts derived from a diallylamine, and those corresponding to the following formula:

H₂C═C(R₁)-D-N+R₂R₃R₄,X⁻

in which:
R₁ represents a hydrogen atom or a methyl group,
R₂ and R₃, which may be identical or different, represent a hydrogen atom or a linear or branched C₁ to C₄ alkyl group,
R₄ represents a hydrogen atom, a linear or branched C₁-C₄ alkyl group or an aryl group,
D represents the following divalent unit: —(Y)ₙ-(A)- in which:
   Y represents an amide function, an ester (O—C(O) or C(O)—O), a urethane or a urea,
   A represents a linear or branched, cyclic or acyclic C₁ to C₁₀ alkylene group, which may be substituted or interrupted with a divalent aromatic or heteroaromatic group. The alkylene groups may be interrupted with an oxygen atom, a nitrogen atom, a sulfur atom or a phosphorus atom; the alkylene possibly being interrupted with a ketone function, an amide, an ester (O—C(O) or C(O)—O), a urethane or a urea,
   n is an integer ranging from 0 to 1,
X⁻ represents an anionic counterion, for instance a chloride or a sulfate.

Examples of water-soluble cationic monomers that may notably be mentioned include the following compounds, and also the salts thereof: dimethylaminoethyl (meth)acrylate, (meth)acryloyloxyethyltrimethylammonium (meth)acrylate, (meth)acryloyloxyethyldimethylbenzylammonium (meth)acrylate, N-[dimethylaminopropyl](meth)acrylamide (meth)acrylate, (meth)acrylamidopropyltrimethylammonium (meth)acrylate, (meth)acrylamidopropyldimethylbenzyl-ammonium (meth)acrylate, dimethylaminohydroxypropyl (meth)acrylate, (meth)acryloyloxyhydroxypropyltrimethylammonium (meth)acrylate, (meth)acryloyl-oxyhydroxypropyldimethylbenzylammonium (meth)acrylate and dimethyldiallylammonium (meth)acrylate.

Among the water-soluble polymers that may be used according to the present invention, mention may also be made of polyhydroxyalcohol (PHA).

Preferably, the water-soluble polymers are polymerized from one or more monomers chosen from vinyl alcohol of formula CH₂═CHOH, vinyl acetate of formula CH₂═CHOC(O)CH₃ and mixtures thereof.

The water-soluble polymers that are capable of forming the envelope of the packaging article may also be chosen from water-soluble polymers derived from natural products, such as polysaccharides, i.e. polymers bearing sugar units. These water-soluble polymers are different from the cationic polysaccharide(s) (v) present in the anhydrous solid composition.

The term “sugar unit” means a unit derived from a carbohydrate of formula Cₙ(H₂O)ₙ₋₁ or (CH₂O)ₙ, which may be optionally modified by substitution and/or by oxidation and/or by dehydration. The sugar units that may be included in the composition of the polymers of the invention are preferably derived from the following sugars: glucose, galactose, arabinose, rhamnose, mannose, xylose, fucose, fructose, anhydrogalactose, galacturonic acid, glucuronic acid, mannuronic acid, galactose sulfate, anhydrogalactose sulfate.

The polymers bearing sugar unit(s) according to the invention may be of natural or synthetic origin. They may be nonionic, anionic, cationic or amphoteric. The base units of the polymers bearing a sugar unit of the invention may be monosaccharides or disaccharides.

As polymers that may be used, mention may notably be made of the following native gums, and also derivatives thereof:
a) tree or shrub exudates, including:
   acacia gum (branched polymer of galactose, arabinose, rhamnose and glucuronic acid);
   ghatti gum (polymer derived from arabinose, galactose, mannose, xylose and glucuronic acid);
   karaya gum (polymer derived from galacturonic acid, galactose, rhamnose and glucuronic acid);
   gum tragacanth (or tragacanth) (polymer of galacturonic acid, galactose, fucose, xylose and arabinose);
b) gums derived from algae, including:
   agar (polymer derived from galactose and anhydrogalactose);
   alginates (polymers of mannuronic acid and of glucuronic acid);
   carrageenans and furcellerans (polymers of galactose sulfate and of anhydrogalactose sulfate);
c) gums derived from seeds or tubers, including:
   guar gum (polymer of mannose and galactose);
   locust bean gum (polymer of mannose and galactose);
   fenugreek gum (polymer of mannose and galactose);
   tamarind gum (polymer of galactose, xylose and glucose);
   konjac gum (polymer of glucose and mannose), the main constituent of which is glucomannan, which is a polysaccharide of high molecular weight (500 000<M_{glucomannan}<2 000 000) composed of D-mannose and D-glucose units with a branch every 50 or 60 units approximately;
d) microbial gums, including:
   xanthan gum (polymer of glucose, mannose acetate, mannose/pyruvic acid and glucuronic acid);
   gellan gum (polymer of partially acylated glucose, rhamnose and glucuronic acid);
   scleroglucan gum (glucose polymer);
   biosaccharide gum (polymer of galacturonic acid, fucose and D-galactose),
e) plant extracts, including:
   cellulose (glucose polymer);
   starch (glucose polymer);
   inulin (polymer of fructose and glucose).

These polymers may be physically or chemically modified. A physical treatment that may notably be mentioned is the temperature. Chemical treatments that may be mentioned include esterification, etherification, amidation and oxidation reactions. These treatments can lead to polymers that may be nonionic, anionic, cationic or amphoteric.

Preferably, these chemical or physical treatments are applied to guar gums, locust bean gums, starches and celluloses.

The nonionic guar gums that may be used according to the invention may be modified with C₁ to C₆ hydroxyalkyl groups. Among the hydroxyalkyl groups, mention may be made of hydroxymethyl, hydroxyethyl, hydroxypropyl and hydroxybutyl groups.

These guar gums are well known in the prior art and may be prepared, for example, by reacting corresponding alkene oxides, for instance propylene oxides, with the guar gum so as to obtain a guar gum modified with hydroxypropyl groups.

The degree of hydroxyalkylation preferably ranges from 0.4 to 1.2 and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The guar gums modified with cationic groups that may more particularly be used according to the invention are guar gums including trialkylammonium cationic groups. Preferably, 2% to 30% by number of the hydroxyl functions of these guar gums bear trialkylammonium cationic groups. Even more preferentially, 5% to 20% by number of the hydroxyl functions of these guar gums are branched with trialkylammonium cationic groups. Among these trialkylammonium groups, mention may most particularly be made of the trimethylammonium and triethylammonium groups. Even more preferentially, these groups represent from 5% to 20% by weight relative to the total weight of the modified guar gum.

According to the invention, guar gums modified with 2,3-epoxypropyltrimethylammonium chloride may be used.

These guar gums modified with cationic groups are products already known per se and are, for example, described in patents U.S. Pat. Nos. 3,589,578 and 4,013,307. Such products are moreover notably sold under the trade names Jaguar C13S, Jaguar C15 and Jaguar C17 by the company Rhodia Chimie.

As modified locust bean gum, use may be made of cationic locust bean gum containing hydroxypropyltrimonium groups, such as Catinal CLB 200 sold by the company Toho.

The starch molecules used in the present invention may originate from any plant source of starch, notably cereals and tubers; more particularly, they may be starches from corn, rice, cassava, barley, potato, wheat, sorghum, pea, oat or tapioca. It is also possible to use hydrolysates of the starches mentioned above. The starch is preferably derived from potato.

The starches may be chemically or physically modified, notably by one or more of the following reactions: pregelatinization, oxidation, crosslinking, esterification, etherification, amidation, heat treatments.

More particularly, these reactions may be performed in the following manner:
pregelatinization by splitting the starch granules (for example drying and cooking in a drying drum);
oxidation with strong oxidizing agents, leading to the introduction of carboxyl groups into the starch molecule and to depolymerization of the starch molecule (for example by treating an aqueous starch solution with sodium hypochlorite);
crosslinking with functional agents capable of reacting with the hydroxyl groups of the starch molecules, which will thus be bonded together (for example with glyceryl and/or phosphate groups);
esterification in alkaline medium for the grafting of functional groups, notably C₁ to C₆ acyl (acetyl), C₁ to C₆ hydroxyalkyl (hydroxyethyl or hydroxypropyl), carboxymethyl or octenylsuccinic.

Monostarch phosphates (of the type St-O—PO—(OX)₂), distarch phosphates (of the type St-O—PO—(OX)—O-St) or even tristarch phosphates (of the type St-O—PO—(O-St)₂) or mixtures thereof may notably be obtained by crosslinking with phosphorus compounds; with St meaning starch and X notably denoting alkali metals (for example sodium or potassium), alkaline-earth metals (for example calcium or magnesium), ammonia salts, amine salts such as salts of monoethanolamine, diethanolamine, triethanolamine or 3-amino-1,2-propanediol, and ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

Distarch phosphates or compounds rich in distarch phosphate may notably be mentioned, for instance the product sold under the references Prejel VA-70-T AGGL (gelatinized hydroxypropyl cassava distarch phosphate), Prejel TK1 (gelatinized cassava distarch phosphate) and Prejel 200 (gelatinized acetylated cassava distarch phosphate) by the company Avebe, or Structure Zea from National Starch (gelatinized corn distarch phosphate).

A preferred starch is a starch that has undergone at least one chemical modification such as at least one esterification.

According to the invention, use may also be made of amphoteric starches, comprising one or more anionic groups and one or more cationic groups. The anionic and cationic groups may be bonded to the same reactive site of the starch molecule or to different reactive sites; they are preferably bonded to the same reactive site. The anionic groups may be of carboxylic, phosphate or sulfate type, preferably carboxylic. The cationic groups may be of primary, secondary, tertiary or quaternary amine type.

The amphoteric starches are notably chosen from the compounds having the following formulae:

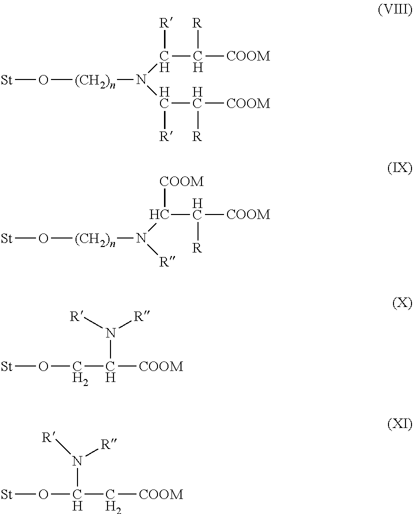

in which formulae (VIII) to (XI):
St-O represents a starch molecule;
R, which may be identical or different, represents a hydrogen atom or a methyl radical;
R′, which may be identical or different, represents a hydrogen atom, a methyl radical or a —C(O)—OH group;
n is an integer equal to 2 or 3;
M, which may be identical or different, denotes a hydrogen atom, an alkali metal or alkaline-earth metal such as Na, K or Li, a quaternary ammonium NH₄, or an organic amine; and
R″ represents a hydrogen atom or a C₁-C₁₈ alkyl radical.

These compounds are notably described in patents U.S. Pat. Nos. 5,455,340 and 4,017,460.

Starches of formula (IX) or (X), and preferentially starches modified with 2-chloroethylaminodipropionic acid are particularly used, i.e. starches of formula (IX) or (X) in which R, R′, R″ and M represent a hydrogen atom and n is equal to 2.

Preferably, the amphoteric starch is a starch chloroethylamido dipropionate.

The celluloses and cellulose derivatives may be anionic, cationic, amphoteric or nonionic.

Among these derivatives, cellulose ethers, cellulose esters and cellulose ester ethers are distinguished.

Among the cellulose esters, mention may be made of inorganic esters of cellulose (cellulose nitrates, sulfates or phosphates), organic esters of cellulose (cellulose monoacetates, triacetates, amidopropionates, acetatebutyrates, acetatepropionates or acetatetrimellitates), and mixed organic/inorganic esters of cellulose, such as cellulose acetatebutyrate sulfates and cellulose acetatepropionate sulfates.

Among the cellulose ester ethers, mention may be made of hydroxypropylmethylcellulose phthalates and ethylcellulose sulfates.

Among the nonionic cellulose ethers that may be mentioned are alkylcelluloses such as methylcelluloses and ethylcelluloses (for example Ethocel Standard 100 Premium from Dow Chemical); hydroxyalkylcelluloses such as hydroxymethylcelluloses and hydroxyethylcelluloses (for example Natrosol 250 HHR sold by Aqualon) and hydroxypropylcelluloses (for example Klucel EF from Aqualon); mixed hydroxyalkyl-alkylcelluloses such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Among the anionic cellulose ethers, mention may be made of carboxyalkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and also the sodium salts thereof.

Among the cationic cellulose ethers, mention may be made of crosslinked or non-crosslinked quaternized hydroxyethylcelluloses. The quaternizing agent may notably be diallyldimethylammonium chloride (for example Celquat L200 from National Starch). Another cationic cellulose ether that may be mentioned is hydroxypropyltrimethylammonium hydroxyethyl cellulose (for example Ucare Polymer JR 400 from Amerchol).

Among the associative polymers bearing sugar unit(s), mention may be made of celluloses or derivatives thereof, modified with groups including at least one fatty chain such as alkyl, arylalkyl or alkylaryl groups or mixtures thereof, in which the alkyl groups are C8-C22; nonionic alkylhydroxyethylcelluloses such as the products Natrosol Plus Grade 330 CS and Polysurf 67 (C₁₆ alkyl) sold by the company Aqualon; quaternized alkylhydroxyethylcelluloses (cationic) such as the products Quatrisoft LM 200, Quatrisoft LM-X 529-18-A, Quatrisoft LM-X5 29-18-B (C₁₂ alkyl) and Quatrisoft LM-X 529-8 (C₁₈ alkyl) sold by the company Amerchol, the products Crodacel QM, Crodacel QL (C₁₂ alkyl) and Crodacel QS (C₁₈ alkyl) sold by the company Croda and the product Softcat SL 100 sold by the company Amerchol; nonionic nonoxynylhydroxyethylcelluloses such as the product Amercell HM-1500 sold by the company Amerchol; nonionic alkylcelluloses such as the product Bermocoll EHM 100 sold by the company Berol Nobel.

As associative polymers bearing sugar unit(s) derived from guar, mention may be made of hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

The water-soluble polymer(s) bearing sugar unit(s) that may be used to form the envelope of the packaging article are preferably chosen from guar gums, locust bean gums, xanthan gums, starches and celluloses, in their modified (derived) form or unmodified form.

Preferably, said polymer(s) bearing sugar unit(s) are nonionic.

The water-soluble polymers described above more particularly have a weight-average molecular weight (Mw) of greater than 1 000 000 and preferably between 1 000000 and 50 000 000. The molecular weight is determined by the RSV (Reduced Specific Viscosity) method as defined in “Principles of Polymer Chemistry” Cornell University Press, Ithaca, N Y 1953 Chapter VII “Determination of Molecular Weight” pages 266-316.

The water-soluble or liposoluble compound(s) that are capable of forming the envelope of the packaging article according to the invention may be in fibre or film form.

According to a first embodiment, the water-soluble or liposoluble compound(s) are in the form of fibres. The term “fibre” refers to any object whose length is greater than its cross section. In other words, it should be understood as referring to an object of length L and of diameter D such that L is greater and preferably very much greater (i.e. at least three times greater) than D, D being the diameter of the circle in which the cross section of the fibre is inscribed. In particular, the ratio L/D (or aspect ratio) is chosen in the range extending from 3.5 to 2500, preferably from 5 to 500, and better still from 5 to 150. The cross section of a fibre may be of any shape: round, serrated or crenellated, or else bean-shaped, but also multilobal, in particular trilobal or pentalobal, X-shaped, in strip form, square, triangular, elliptical or the like. The fibres of the invention may or may not be hollow.

According to this embodiment, the fibres may be spun, carded or twisted. Advantageously, the fibres used in the context of the present invention are spun. The mean diameter of the fibres used according to the present invention, which may be identical or different, is less than 500 μm. Advantageously, such a diameter is less than 200 μm, preferably less than 100 μm, or even less than 50 μm.

Mention may be made more particularly of water-soluble fibres which include fibres based on PVA (polyvinyl alcohol), fibres of polysaccharides such as glucomannans, starches, celluloses such as carboxymethylcelluloses, polyalginic acid fibres, polylactic acid fibres and polyalkylene oxide fibres, and also mixtures thereof. More preferentially, the water-soluble fibre(s) used in the invention are chosen from PVA-based fibres.

The fibres of the envelope are generally entangled. The term “envelope comprising water-soluble fibres” means an envelope which may consist entirely of water-soluble fibres which may include both fibres that are water-soluble and fibres that are water-insoluble at a temperature of less than or equal to 35° C., the soluble fibres needing to be in larger amount than the insoluble fibres. The envelope of the fibres must include at least 60% by weight of soluble fibres, preferably at least 70% and better still at least 80% by weight relative to the total weight of the fibres. It may thus include, for example, more than 95% by weight, or even more than 99% by weight and even 100% by weight of water-soluble fibres relative to the total weight of the fibres of the envelope.

When the envelope contains insoluble fibres, these may be made of any material commonly used as insoluble fibres; they may be, for example, silk, cotton, wool, flax, polyamide (Nylon®), polylactic acid, modified cellulose (rayon, viscose, rayon acetate), poly-p-phenylene terephthalamide, notably Kevlar®, polyolefin and notably polyethylene or polypropylene, glass, silica, aramid, carbon, notably in graphite form, Teflon®, insoluble collagen, polyester, polyvinyl chloride or polyvinylidene chloride or polyethylene terephthalate fibres, or fibres formed from a mixture of the compounds mentioned above, such as polyamide/polyester or viscose/polyester fibres.

In addition, when the envelope contains fibres, it may be woven or nonwoven.

According to a first variant of the invention, the envelope may be woven. In the context of the present invention, a “woven” material results from an organized assembly of fibres, in particular of water-soluble polymeric fibres, and more particularly of an intercrossing, in the same plane, of said fibres, arranged in the direction of the warp and of fibres arranged, perpendicular to the warp fibres, in the direction of the weft. The bonding obtained between these warp and weft fibres is defined by a weave.

Such a woven material results from an operation directed towards assembling the fibres in an organized manner such as weaving per se, but may also result from knitting.

According to another variant of the invention, the envelope is nonwoven.

For the purposes of the present invention, the term “nonwoven fabric” refers to a substrate comprising fibres, in particular water-soluble polymeric fibres, in which the individual fibres are arranged in a disordered manner in a structure in the form of a lap and which are neither woven nor knitted. The fibres of the nonwoven fabric are generally bonded together, either under the effect of a mechanical action (for example needle punching, air jet or water jet), or under the effect of a thermal action, or by addition of a binder.

Such a nonwoven fabric is, for example, defined by the standard ISO 9092 as a web or lap of directionally or randomly oriented fibres, bonded by friction and/or cohesion and/or adhesion, excluding paper and products which are woven, knitted, tufted or stitch-bonded incorporating bonding yarns or filaments.

A nonwoven fabric differs from a paper by the length of the fibres used. In paper, the fibres are shorter. However, there are nonwoven fabrics based on cellulose fibre, which are manufactured by a wet-laid process and which have short fibres like in paper. The difference between a nonwoven fabric and a paper is generally the absence of hydrogen bonding between the fibres in a nonwoven fabric.

Very preferentially, the fibres used in the context of the present invention are chosen from synthetic fibres such as PVA fibres. In particular, the envelope is nonwoven, and is preferentially made of nonwoven PVA fibres.

To make the nonwoven envelope of the packaging article, use is preferably made of PVA fibres that are soluble in water at a temperature of less than or equal to 35° C., for instance the fibres sold by the Japanese company Kuraray under the name Kuralon K-II, and particularly the grade WN2 which is soluble at and above 20° C. These fibres are described in EP-A-636 716 which teaches the manufacture of PVA fibres that are soluble in water at temperatures not exceeding 100° C., by spinning and drawing of the wet or dry polyvinyl alcohol polymer in the presence of solvents participating in the dissolution and solidification of the fibre. The fibre thus obtained may lead to the production of woven or nonwoven substrates.

These fibres may also be prepared from a solution to be spun, by dissolving a water-soluble PVA-based polymer in a first organic solvent, spinning of the solution in a second organic solvent to obtain solidified filaments and wet drawing of the filaments, from which the first solvent is removed, followed by drying and subjecting to a heat treatment. The cross section of these fibres may be substantially circular. These fibres have a tensile strength of at least 2.7 g/dtex (3 g/d). Patent application EP-A-0 636 716 describes such water-soluble PVA-based fibres and the process for manufacturing them. For example, the fibres may also be formed by extrusion and deposited on a conveyor to form a lap of fibres which is then consolidated via a conventional fibre bonding technique, for instance needle punching, hot bonding, calendering or air-through bonding, in which technique the water-soluble lap passes through a tunnel into which hot air is blown, or spunlacing directed towards bonding the fibres under the action of fine jets of water at very high pressure, which cannot be applied to fibres whose dissolution temperature is too low.

As has been seen previously, the invention is not limited to the use of PVA, and use may also be made of fibres made from other water-soluble materials provided that these materials dissolve in water having the desired temperature, for example the polysaccharide fibres sold under the name Lysorb by the company Lysac Technologies Inc. or other fibres based on polysaccharide polymers such as glucomannans or starch.

The envelope may comprise a mixture of different fibres that are soluble in water at different temperatures (up to 35° C.).

The fibres may be composite, and they may include, for example, a core and a sheath which are not of the same nature, for example formed from different grades of PVA.

According to a particular embodiment of the invention, the envelope is a nonwoven fabric, including water-soluble fibres, alone or as a mixture with insoluble fibres as indicated above, with not more than 40% by weight of insoluble fibres relative to the total weight of the fibres constituting the lap. Preferably, the nonwoven fabric consists essentially of water-soluble fibres, i.e. it does not contain any insoluble fibres.

According to another embodiment of the invention, the envelope of the packaging article may consist of one or more films, which each comprise one or more water-soluble and/or liposoluble compounds, notably as defined above. When the envelope consists of several films, said films may be assembled, for example bonded together, so as to form a single unified film.

The thickness of the “overall” film (i.e. the thickness of the single film when the envelope contains only one or of the unified film when the envelope contains several films) is advantageously between 10 and 1000 microns, preferably between 10 and 800 microns and more preferentially between 15-500 microns.

The term “film” notably means a continuous layer preferentially formed from one or more water-soluble and/or liposoluble compounds as defined above, in particular of polymer(s).

The main industrial methods for the production of polymer films are extrusion of a molten polymer, casting of a solution of a polymer onto a polished metal surface (in certain cases, the polymer solution is introduced into a precipitation tank), casting of a dispersion of the polymer onto a polished surface, and calendering.

The films that may be used according to the present invention may be chosen from film-multilayer film, film-paper (laminating) and film-coating.

During application by spraying, brushing or various industrial processes, the surface coatings undergo what is known as the formation of a film, and notably of film-coating. In the majority of the film-forming processes, a liquid coating of relatively low viscosity is applied to a solid substrate and is hardened as a solid adherent film based on high molecular weight polymer having the properties desired by the user.

The films that may be used according to the present invention are notably PVA films which may be manufactured via any industrial production method, such as a method of casting a PVA-based polymer solution, a method of extrusion in the presence or absence of water, a dry-extrusion moulding method or a biaxial orientation method.

The packaging article, and the envelope, may have any shape that is suitable for the intended use, for example a rectangular, round or oval shape. Preferably, it has a rounded geometry, for example in the form of a sphere, a disc or an oval, or else a square or parallelepipedal geometry preferably with rounded corners. The envelope preferably has dimensions allowing it to be taken up between at least two fingers. Thus, it may, for example, have an ovoid shape about 2 to 10 cm long and about 0.5 to 4 cm wide, or a circular disc shape about 2 to 10 cm in diameter, or a square shape with a side length of about 2 to 15 cm, or a rectangular shape with a length of about 2 to 25 cm, it being understood that it may have any other shape and size that are suitable for the intended use.

Preferably, the envelope may be of round shape with an inside diameter ranging from 3 to 7 cm, more preferentially from 4 to 5 cm; to which may be added the dimension of the edges (sealed part) which may range from 1 to 5 mm, better still from 2 to 4 mm; and a height ranging from 2 to 7 mm, preferentially from 3 to 5 mm.

The envelope may also be of square or rectangular shape with a length preferably ranging from 2 to 6 cm, more preferentially from 3 to 5 cm, and a width preferably ranging from 2 to 5 cm, more preferentially 2.5 to 4 cm; to which may be added the dimension of the edges (sealed part) which may preferably range from 1 to 5 mm, and more preferentially from 2 to 4 mm.

Advantageously, the envelope has a low thickness, and may consist of several layers of different materials. Preferably, the thickness of the envelope ranges from 3% to 99.9% of its other dimensions. The envelope is thus substantially flat, with thin edge profiles.

The area delimiting the cavity or cavities has an extent advantageously less than 625 cm², preferably between 0.025 cm² and 400 cm², more preferentially between 1 and 200 cm², better still between 2 and 50 cm² and even better still between 4 and 25 cm², so as to have optimized compacting of the composition. It has been observed that when the area of the article is within the above ranges, the compacting of the anhydrous solid composition made of powder is lower and the transformation of the powder into a fluid composition in the hands is easier, without any formation of agglomerates.

Preferably, the height of the envelope is greater than or equal to 2 mm, more preferentially ranging from 2 to 10 mm and better still from 3 to 7 mm.

Preferably, the film(s) used in the context of the present invention are chosen from synthetic films such as PVA or PVOH films, and also mixtures thereof.

Preferably, the envelope consists of several layers, for example two or three layers, of films which are each preferably made of different materials. Advantageously, at least one of these films is a film comprising or consisting of PVA and/or PVOH.

Preferably, the film(s) are sealed so as to form one or more cavities which will comprise the anhydrous solid composition of the invention and will prevent it from escaping.

Advantageously, the packaging article comprises from 1 to 5 g and preferably from 2 to 4.5 g of anhydrous solid composition; and from 0.1 to 0.8 g and preferably from 0.2 to 0.5 g of envelope.

The present invention also relates to a cosmetic process for treating keratin fibres, in particular human keratin fibres such as the hair, comprising a step of using a packaging article as defined above; preferably, said cosmetic treatment process comprises the following steps:
i) mixing the packaging article in a composition that is capable of dissolving, totally or partially, the envelope of said packaging article,
ii) applying the composition obtained in step i) to the keratin fibres,
iii) optionally leaving to stand,
iv) rinsing said keratin fibres, and
v) optionally drying said keratin fibres.

It is understood that the composition that is suitable for dissolving the envelope depends on the nature of the envelope. In other words, the composition that is suitable for dissolving the envelope is water or an aqueous composition when the packaging article predominantly or solely contains a hydrophilic envelope. Further, the composition that is suitable for dissolving the envelope is an anhydrous organic composition or an aqueous composition comprising at least one liquid fatty substance or at least one organic solvent other than liquid fatty substances such as lower monoalcohols, for example ethanol, or such as polyols, for example propylene glycol or glycerol, when the packaging article predominantly or solely contains a lipophilic envelope.

Thus, the aqueous composition may simply be water. The aqueous composition may optionally comprise at least one polar solvent. Among the polar solvents that may be used in this composition, mention may be made of organic compounds that are liquid at room temperature (25° C.) and at least partially water-miscible.

Examples that may be mentioned more particularly include alkanols such as ethyl alcohol and isopropyl alcohol, aromatic alcohols such as benzyl alcohol and phenylethyl alcohol, or polyols or polyol ethers, for instance ethylene glycol monomethyl ether, monoethyl ether and monobutyl ether, propylene glycol or ethers thereof, for instance propylene glycol monomethyl ether, butylene glycol, dipropylene glycol, and also diethylene glycol alkyl ethers, for instance diethylene glycol monoethyl ether or monobutyl ether.

More particularly, if one or more solvents are present, their respective content in the aqueous composition ranges from 0.5% to 20% by weight and preferably from 2% to 10% by weight relative to the weight of said aqueous composition.

The dilution ratio (expressed by weight) between one or more packaging articles, as defined previously, and the composition that is suitable for dissolving the packaging article(s) is preferably between 10/90 and 90/10 and more preferentially between 10/90 and 50/50. Better still, this dilution ratio is 20/80.

In particular, the composition obtained on conclusion of the mixing (step i) of the process) may be applied to wet or dry keratin fibres. It is advantageously left in place on the keratin fibres for a time generally ranging from 1 to 15 minutes, preferably from 2 to 10 minutes.

The keratin fibres are then rinsed with water. They may optionally be washed with a shampoo, followed by rinsing with water, before being dried or left to dry.

A subject of the present invention is also the use of a packaging article, as defined previously, for washing and/or conditioning keratin fibres, and in particular human keratin fibres such as the hair.

The examples that follow serve to illustrate the invention without, however, being limiting in nature.

### EXAMPLES

### Example 1

The anhydrous solid composition A according to the invention, below, was prepared from ingredients whose contents are indicated in the table below (% in g of active material).

TABLE 1

| | Composition A |
|---|---|
| Sodium cocoyl | 10.2 |
| isethionate | |
| Sodium lauroyl | 23 |
| glutamate | |
| Cocamidopropyl betaine | 10 |
| Hydroxypropyl guar | 0.6 |
| hydroxypropyltrimonium | |
| chloride | |
| Magnesium stearate | 5 |
| Sodium bicarbonate | 3.75 |
| Citric acid | 1.25 |
| Sodium chloride | 1.8 |
| Fragrance | 3.5 |
| Corn starch | qs 100 |

Composition A thus obtained is in anhydrous powder form (its water content (originating from the starting materials) being equal to 0.17% by weight, relative to the total weight of the composition) and may be used for washing hair.

### Example 2

a) Compositions Tested

The anhydrous solid compositions A1 and A2 according to the invention, below, were prepared from ingredients whose contents are indicated in the table below (% in g of active material).

TABLE 2

| | Composition | Composition |
|---|---|---|
| | A1 | A2 |
| Cocamidopropyl betaine | 10 | 10 |
| Sodium cocoyl | 10.2 | 10.2 |
| isethionate | | |
| Sodium lauroyl | 23 | 23 |
| glutamate | | |
| Hydroxypropyl guar | 0.6 | — |
| hydroxypropyltrimonium | | |
| chloride | | |
| Cationic cellulose | — | 0.6 |
| ether (polyquaternium- | | |
| 67) | | |
| Magnesium stearate | 5 | 5 |
| Sodium bicarbonate | 3.75 | 3.75 |
| Citric acid | 1.25 | 1.25 |
| Fragrance | 3.5 | 3.5 |
| Corn starch | qs 100 | qs 100 |

b) Protocol

The foam stability and the cosmetic properties of each of the resulting compositions A1 and A2 are evaluated by the following methods:

Foam Stability

0.2 g of each of compositions A1 and A2 is weighed out into a plastic cup. Tresses of natural hair each weighing 2.7 g and wetted beforehand (by being passed under tap water at 37° C. 5 times and then wrung out twice) are placed in the cups. The tresses are subsequently massaged for 20 seconds, by performing circular movements in the cup with the fingers. The tresses are subsequently wrung out twice and the resulting foam is collected and photographed.

The tresses are compared with an untreated, wetted tress.

Cosmetic Properties

A panel of 3 experts evaluated the cosmetic properties, with regard to suppleness, smoothing and disentangling, of each of the tresses wrung out, and compared them with an untreated, wetted tress. The testers assigned a score ranging from −3 to +3 to each of the following cosmetic properties: suppleness, smoothing and disentangling, according to the following criteria: negative scores correspond to properties less good than those of the reference, 0 being identical to the reference, and positive scores correspond to properties superior to the reference.

The same scoring system was used to evaluate the foam stability.

c) Results

The results are given in the table below.

TABLE 3

| | Composition A1 | Composition A2 |
|---|---|---|
| Foam stability | +2 | +1 |
| Cosmetic properties | +3 | +1 |
| (average of the 3 experts) | | |

The resulting compositions A1 and A2 are in powder form and can be used for washing hair. They enable rapid foam onset and generate an abundant foam. The foams obtained from compositions A1 and A2 exhibit high stability, and this stability is also improved with composition A1. The foam obtained from composition A1, indeed, is creamier than that from composition A2.

The results above show that the compositions according to the invention also impart good cosmetic properties to the hair, particularly in terms of disentangling, suppleness and smoothing. These properties, moreover, are improved after application of composition A1.

### Example 3

The following anhydrous solid compositions A3, A4 and A5 were prepared, according to the invention, from ingredients whose contents are indicated in the table below (% in g of active material).

TABLE 4

| | Composition | Composition | Composition |
|---|---|---|---|
| | A3 | A4 | A5 |
| Cocamidopropyl | 8.5 | 8.5 | 8.5 |
| betaine | | | |
| Sodium cocoyl | 8.3 | 8.3 | 8.3 |
| isethionate | | | |
| Sodium lauroyl | 16.5 | 16.5 | 16.5 |
| glutamate | | | |
| Hydroxypropyl | 0.2 | 0.4 | 0.8 |
| guar hydroxy- | | | |
| propyltrimonium | | | |
| chloride | | | |
| Cetylstearyl | 1.2 | 1.2 | 1.2 |
| glucoside/cetyl- | | | |
| stearyl alcohol | | | |
| mixture | | | |
| Fragrance | 0.5 | 0.5 | 0.5 |
| Corn starch | qs 100 | qs 100 | qs 100 |

The compositions A3-A5 thus prepared are packaged in powder form into a water-soluble PVOH-based sachet. The packaging article thus obtained can then be used as a washing composition: it is placed in the palm of the hand, water is added to dissolve it and optionally to form a foam, and it is then applied onto the hair, which has preferably been moistened beforehand.

### Example 4

a) Compositions Tested

Composition (A6) according to the invention and comparative composition (B) were prepared from ingredients whose contents are indicated in the table 5 below (% in g of active material).

TABLE 5

| | Composition A6 | Composition B |
|---|---|---|
| | (invention) | (comparative) |
| Sodium cocoyl | 10.2 | 10.2 |
| isethionate | | |
| Sodium lauroyl | 23 | 23 |
| glutamate | | |
| Cocamidopropyl betaine | 10 | 10 |
| Hydroxypropyl guar | 0.6 | — |
| hydroxypropyltrimonium | | |
| chloride | | |
| Magnesium stearate | 5 | 5 |
| Sodium bicarbonate | 3.75 | 3.75 |
| Citric acid | 1.25 | 1.25 |
| Sodium chloride | 1.8 | 1.8 |
| Fragrance | 3.5 | 3.5 |
| Corn starch | qs 100 | qs 100 |

b) Evaluation of the Foam Quality

Each of compositions (A6) and (B) thus obtained were applied to wet hands, before being applied on ½ malleable heads whose hair had been wetted beforehand, at a rate of 2 g of composition per ½ head.

The ½ heads were then massaged 12 seconds with the fingers to generate foam.

The foam quality has then been evaluated visually. The foam obtained with the composition A6 according to the present invention is more abundant, creamier and presents a better hold than the foam obtained with the comparative composition B.

c) Evaluation of the Cosmetic Properties

0.2 g of each of compositions (A6) and (B) thus obtained were applied on wet hands, before being applied on medium sensitized locks of hair (alkaline solubility=20%, SA 20), which had been wetted beforehand.

The locks were subsequently massaged with the fingers for 15 seconds. After a leave-on time of 5 minutes, the locks were rinsed with water at 35° C. for 10 seconds (corresponding to 15 passes between the fingers) and then wrung out.

The level of suppleness conferred to the hair has then been evaluated on wet hair by three experts, in a blind test manner. Each of the experts compared the locks of hair and assigned a score to the lock a suppler touch, assigning a +1 for the lock presenting the best performance level, and +2 when the difference was significant.

The assessment of suppleness (or flexibility) is tactile. The expert takes the lock of hair in his hands and tries to bend it. He evaluates if the hair is easy to bend, if it is malleable.

The results obtained are given in the table below (average value of the scores assigned by the 3 experts).

| | Composition A6 | Composition B |
|---|---|---|
| | invention | comparative |
| suppleness | +2 | — |

The results thus obtained show that composition A6 according to the present invention confers better suppleness to the hair than the comparative composition B3.

Thus, composition A4 is not only able to generate a foam presenting better qualities, that is indeed more abundant, firm and creamy, but also confers better cosmetic properties, notably in terms of suppleness, to the hair than the comparative composition.

1-17. (canceled)

18. A solid composition, comprising:
(i) at least one anionic surfactant of sulfonate type,
(ii) at least one anionic surfactant of carboxylate type,
(iii) at least one amphoteric or zwitterionic surfactant,
(iv) at least one filler, and
(v) at least cationic polysaccharide other than the at least one filler (iv), and
water in a total amount of less than 5% by weight, relative to the total weight of the composition.

19. The composition of claim 18, wherein the at least one anionic surfactant of sulfonate type is chosen from alkylsulfonates, alkylamidesulfonates, alkylarylsulfonates, alpha-olefinsulfonates, paraffinsulfonates, alkylsulfosuccinates, alkylethersulfosuccinates, alkylamidesulfosuccinates, alkylsulfoacetates, sulfolaurates, N-acyltaurates, acylisethionates, salts thereof, or mixtures of two or more thereof.

20. The composition of claim 19, wherein the at least one anionic surfactant is chosen from polyoxyalkylenated compounds.

21. The composition of claim 18, wherein the at least one anionic surfactant of sulfonate type is chosen from compounds of formula (I):
R₁—COX—R₂—SO₃M (I)
wherein in formula (I):
R₁ represents a linear or branched alkyl group comprising from 8 to 30 carbon atoms,
X represents an oxygen atom, a —N(CH₃)—, or a —NH— group,
R₂ represents a linear or branched alkyl group comprising from 1 to 4 carbon atoms, and
M represents a hydrogen atom, an ammonium ion, an ion derived from an alkali metal or alkaline-earth metal, or an ion derived from an organic amine.

22. The composition of claim 18, wherein the total amount of the anionic surfactant(s) of sulfonate type (i) ranges from 1% to 30% by weight, relative to the total weight of the composition.

23. The composition of claim 18, wherein the at least one anionic surfactant of carboxylate type is chosen from compounds of formula (II):
R—(OCH₂CH₂)ₙW—(CHY₁)ₚ—COOX (II),
wherein in formula (II):
Y₁ represents a hydrogen atom, a (CH₂)_{q}COOX group, or a hydroxyl group;
W represents an oxygen atom, a (O-Glu-O)ᵣ—(COCH(Y₂)—(C(OH)COOX)ₜ)ₛ group, or a CO—NR₃ group;
Y₂ represents a hydrogen atom or a hydroxyl group;
R₃ represents a hydrogen atom or a methyl group;
X represents a hydrogen atom, an ammonium ion, an ion derived from an alkali metal or alkaline-earth metal, or an ion derived from an organic amine;
R represents a linear or branched alkyl group comprising from 8 to 30 carbon atoms;
Glu represents a divalent radical derived from glucopyranose by removal of 2 hydroxyl groups;
p is equal to 0 or 1;
q denotes an integer ranging from 1 to 10;
n denotes an integer ranging from 0 to 50;
r denotes a number ranging from 1 to 10;
s is equal to 0 or 1; and
t is equal to 0 or 1.

24. The composition of claim 18, wherein the at least one anionic surfactant of carboxylate type is chosen from N-acylglycinates,
N-acylsarcosinates, and
N-acylglutamates.

25. The composition of claim 18, wherein the total amount of anionic surfactant(s) of carboxylate type ranges from 1% to 40% by weight, relative to the total weight of the composition.

26. The composition of claim 18, wherein the weight ratio of the total amount of anionic surfactant(s) of carboxylate type to the total amount of anionic surfactant(s) of sulfonate type is greater than or equal to 3:5.

27. The composition of claim 18, wherein the at least one amphoteric or zwitterionic surfactant is chosen from alkyl(C₈-C₂₀)betaines, alkyl(C₈-C₂₀)amidoalkyl(C₃-C₈)betaines, or mixtures of two or more thereof.

28. The composition of claim 18, wherein the at least one filler is chosen from polymeric organic fillers or mixtures of two or more thereof.

29. The composition of claim 28, wherein the at least one filler is chosen from cyclodextrins, starches, alginates, gellans, guar gums, celluloses, wood flours, crosslinked polyvinylpyrrolidones, polyacrylates, or mixtures of two or more thereof.

30. The composition of claim 18, wherein the total amount of filler(s) is at least 20% by weight, relative to the total weight of the composition.

31. The composition of claim 18, wherein the total amount of filler(s) is at least 35% by weight, relative to the total weight of the composition.

32. The composition of claim 18, wherein the at least one cationic polysaccharide other than the at least one filler is chosen from cationic galactomannan gums or mixtures of two or more thereof.

33. The composition of claim 18, wherein the total amount of the cationic polysaccharide(s) is at least 0.05% by weight, relative to the total weight of the composition.

34. The composition of claim 18, wherein the total amount of the cationic polysaccharide(s) ranges from 0.1% to 2% by weight, relative to the total weight of the composition.

35. A cosmetic method for treating keratin fibers, comprising applying to the keratin fibers a solid composition comprising:
(i) at least one anionic surfactant of sulfonate type,
(ii) at least one anionic surfactant of carboxylate type,
(iii) at least one amphoteric or zwitterionic surfactant,
(iv) at least one filler,
(v) at least cationic polysaccharide other than the at least one filler (iv), and
water in a total amount of less than 5% by weight, relative to the total weight of the composition,
wherein the solid composition is moistened with water, then applied to the keratin fibers.

36. A packaging article comprising:
at least one cavity,
a solid composition comprising:
(i) at least one anionic surfactant of sulfonate type,
(ii) at least one anionic surfactant of carboxylate type,
(iii) at least one amphoteric or zwitterionic surfactant,
(iv) at least one filler,
(v) at least cationic polysaccharide other than the at least one filler (iv), and
water in a total amount of less than 5% by weight, relative to the total weight of the composition,
wherein the solid composition is disposed in one of the cavities of the packaging article, and
wherein a material of the packaging article comprises one or more water-soluble and/or liposoluble compounds.

37. A cosmetic method for treating keratin fibers, comprising:
i) mixing the packaging article according to claim 36 in a composition that capable of dissolving the packaging article,
ii) applying the composition obtained in step i) to the keratin fibers,
iii) rinsing the keratin fibers, and
iv) optionally drying the keratin fibers.
